(19) United States
(12) Patent Application Publication
(10) Pub. No.: US 2003/0224019 A1
(43) Pub. Date: Dec. 4, 2003

(54) Methods of treating nerve entrapment syndromes

(76) Inventor: Christopher O'Brien, San Diego, CA (US)

Correspondence Address: BURNS DOANE SWECKER & MATHIS L L P, POST OFFICE BOX 1404, ALEXANDRIA, VA, 22313-1404, US

(21) Appl. No.: 10/378,042

(22) Filed: Feb. 27, 2003

Related U.S. Application Data
(60) Provisional application No. 60/360,628, filed on Mar. 1, 2002.

(51) Int. Cl. A61K 39/08

(52) U.S. Cl. 424/239.1

(57) ABSTRACT

Methods of treating nerve entrapment syndromes and/or the pain associated with nerve entrapment syndromes using botulinum type B toxin are disclosed. These methods involve, for example, injections of botulinum type B toxin to the tissue impinging on the nerve and/or the interstitial area around the nerve and/or the connective tissue surrounding the nerve.

### CROSS-REFERENCE TO RELATED APPLICATIONS

This application claims the benefit of U.S. Provisional Application Serial No. 60/360,628, filed on Mar. 1, 2002, which is incorporated herein by reference in its entirety.

### FIELD OF THE INVENTION

The invention relates to a method for the alleviation of pain in treatment of nerve entrapment syndromes by the use of botulinum type B toxin.

### BACKGROUND OF THE ART

Botulinum toxin is a polypeptide product of the anaerobic bacterium Clostridium botulinum. The toxin causes muscle paralysis in mammals by blocking presynaptic release of the neurotransmitter acetylcholine at the neuromuscular junction. While the toxin has long been associated with fatal botulism, in recent years it has become a new therapeutic modality for certain neuromuscular disorders and has gained rapid acceptance and expanding usage. For example, serotype A of the Botulinum toxin has been recommended in the art for use for the treatment of certain diseases, such as disorders of the extraocular muscles (e.g., comitant strabismus and nystagmus) as well as dystonias (involuntary contractions of facial muscle, e.g. hemifacial spasm) (see, e.g., The New England Journal of Medicine, 324:1186-1194, 1991). The toxin is administered in a pharmaceutically safe form directly into affected muscles, usually via injection, however iontophoresis and other methods of administration are available. The advantage of using Botulinum toxin A in this context is that it produces a reversible, flaccid paralysis of mammalian skeletal muscle, presumably by blocking the exocytosis of acetylcholine at peripheral, presynaptic cholinergic receptors, with limited activity at receptors in the central nervous system (Rabasseda, et al., Toxicon, 26:329-326, 1988). Additionally, Botulinum toxin A is not believed to result in degeneration of nervous or muscular tissue and has been approved for use in certain therapies by the Food and Drug Administration.

Other serotypes of the Botulinum toxin have been identified that have immunologically distinct phenotypes; i.e., serotypes B, C1, C2, D, F and G (Simpson, et al., Pharmacol.Rev., 33:155-188, 1981). Botulinum toxin type B is available as Myoblock™ in the United States and is available as a stable liquid, sterile formulation and also has FDA approval for treatment of cervical dystonia. All of the serotypes are believed to be proteins of about 150 kDa molecular weight that are comprised of two polypeptide chains linked by disulfide bridges. The shorter of the two chains is believed to be responsible for the toxicity of the toxin, while the longer of the two chains is believed to be responsible for the penetration of the toxin into nervous tissue. Each toxin type is antigenically distinct and thus described as serotypes.

A journal article entitled CT-guided (computed tomagraphy) injection of botulinic toxin for percutaneous therapy of piriformis muscle syndrome with preliminary MRI results about denervative process. Fanucci E; et al., European Radiology (Germany) 2001, 11 (12) p2543-8 has described the use of Botulinum toxin type A in treatment of piriformis syndrome.

### DESCRIPTION OF THE INVENTION

Nerve entrapment syndromes involve the trapping or compression of a peripheral nerve, either by muscle, vascular, skeletal or connective tissues. This entrapment and compression or deformation causes a variety of painful symptoms from shooting pain to numbness and tingling. Traditionally the physician advised, in the case of those that are brought on by movement, the cessation of that movement, whether it be a repetitive movement or one that is performed during sporting activities. In the arm and wrist, the affected limb may be immobilized by the use of a splint or sling. Anti-inflammatories are also administered to lessen inflammation and swelling which exacerbates nerve compression. If these conservative approaches are not successful, the next step is usually surgical intervention.

It has now been surprisingly found that botulinum toxin type B is not only effective treating nerve compression diseases caused by muscle tissue impinging on the affected nerve, but also in nerve compression syndromes where the nerve is predominantly surrounded by other types of tissue. While not wishing to be held to a particular theory, applicants theorize that the type B toxin not only produces temporary flaccid muscle paralysis, but also has a pain blocking effect as well. Thus botulinum toxin type B is a superior therapeutic in treating not only nerve compression caused by muscle tissue, but by vascular, connective and bone tissue as well.

Specific nerve entrapment syndromes are hereinafter described by way of providing non-limiting examples:

Carpal Tunnel Syndrome

Compression of the Median Nerve as it Passes Through the Carpal Tunnel in the Wrist.

Carpal tunnel syndrome is very common and most commonly occurs in women aged 30 to 50 yr. Causes include RA (Rheumatoid Arthritis, sometimes the presenting manifestation), diabetes mellitus, hypothyroidism, acromegaly, amyloidosis, and pregnancy (producing edema in the carpal tunnel). Activities or jobs that require repetitive flexion and extension of the wrist (eg, keyboard use) may pose an occupational risk. Often, no underlying cause can be found.

Symptoms and Diagnosis

Symptoms include pain of the hand and wrist associated with tingling and numbness, classically distributed along the median nerve (the palmar side of the thumb, the index and middle fingers, and the radial half of the ring finger) but possibly involving the entire hand. Typically, the patient wakes at night with burning or aching pain and with numbness and tingling and shakes the hand to obtain relief and restore sensation.

Diagnosis is indicated by a positive Tinel's sign, in which the tingling (paresthesia) is reproduced by tapping with a reflex hammer at the volar surface of the wrist over the site of the median nerve and carpal tunnel. Additional tests include wrist flexion maneuvers (eg, Phalen's sign). Thenar atrophy and weakness on thumb elevation may develop later. A diagnosis is typically confirmed by electrodiagnostic testing of median nerve conduction velocity, which provides an accurate index of motor and sensory nerve conduction.

Previous Treatment Modalities

Treatment includes a lightweight wrist splint, especially at night; possibly pyridoxine (vitamin B₆) 50 mg bid; and mild analgesics (eg, acetaminophen, NSAIDs (non-steroidal anti-inflammatory drugs)). Some persons find relief by changing the position of computer keyboards and making other ergonomic corrections. If these measures fail to control symptoms, a corticosteroid should be locally injected into the carpal tunnel at a site just ulnar to the palmaris longus tendon and proximal to the distal crease at the wrist. If bothersome symptoms persist or recur or if hand weakness and thenar wasting progress, surgical decompression of the carpal tunnel using an open technique or endoscopy is recommended.

Cubital Tunnel Syndrome (Ulnar Neuropathy)

Compression of the Ulnar Nerve at the Elbow, Resulting in Numbness and Paresthesia of the Ring and Little Fingers.

Cubital tunnel syndrome is less common than carpal tunnel syndrome. Baseball pitchers are prone to cubital tunnel syndrome because of the extra twist of the arm required to throw a slider. Symptoms include numbness and paresthesia on the ulnar side of the hand and elbow pain. The ulnar nerve passes around the elbow, and anyone who has ever banged his or her funny bone knows how sensitive this nerve can be. This nerve may become chronically inflamed and entrapped in its tight passage around the elbow (the passage is called the cubital tunnel). In advanced stages, weakness of the ring and little fingers may develop. It is differentiated from ulnar nerve entrapment at the wrist (ie, in Guyon's canal) by sensory testing, location of Tinel's sign, and electromyography and nerve conduction velocity testing. Weakness interferes with pinch of the thumb and index finger. Traditional treatment has involved splinting at night, with the elbow partially extended, and possibly empirical administration of pyridoxine (vitamin B₆) 50 mg po bid. Surgical decompression was previously considered the only alternative if conservative treatment failed.

Radial Tunnel Syndrome (Posterior Interosseous Nerve Syndrome)

Compression of the Superficial Branch of the Radial Nerve in the Proximal Forearm or Back of the Arm, Resulting in Lancinating Pain into the Dorsum of the Forearm and Hand.

Lesions at the elbow level include trauma, ganglia, lipomas, bone tumors, and radial bursitis. Pain is precipitated by attempted extension of the wrist and fingers. There is no sensory loss because the radial nerve is principally a motor nerve. Localized Tinel's sign and tenderness along the course of the radial nerve must be distinguished from lateral epicondylitis. Avoiding the forceful or repeated motion of supination or dorsiflexion reduces pressure on the nerve and permits resolution of the manifestations. If wristdrop develops, surgical decompression may be needed.

Piriformis Syndrome

Sciatic pain can be caused by compression of the sciatic nerve by the piriformis muscle. This condition is commonly referred to as sciatica and is quite common in the middle-aged and elderly. The piriformis muscle extends from the pelvic surface of the sacrum to the upper border of the greater trochanter of the femur and, during running or sitting, can squeeze the sciatic nerve at the site where the nerve emerges from under the piriformis to over the gemellus and obturator internus muscles.

Symptoms and Signs

A chronic nagging ache, pain, tingling, or numbness starts in the buttocks but can extend along the course of the sciatic nerve, down the entire back of the femur and tibia, and in front of the tibia. Pain is usually chronic and worsens when the piriformis is pressed against the sciatic nerve (eg, while sitting on a toilet, a car seat, or a narrow bicycle seat or while running). Unlike piriformis pain, disk compression of the sciatic nerve is usually associated with lumbar pain, particularly during lumbar extension.

Diagnosis

Thorough physical examination is essential for diagnosis: Freiberg's maneuver (forceful internal rotation of the extended thigh) stretches the piriformis muscle, causing pain. Pace's maneuver (abducting the affected leg) elicits pain in a sitting patient. For Beatty's maneuver, the patient lies on a table on the side of the nonaffected leg. The affected leg is placed behind the nonaffected leg with the bent knee on the table. Raising the knee several inches off the table causes pain in the buttocks. For the Mirkin test, the patient should stand, keeping the knees straight, and slowly bend toward the floor. The examiner should press into the buttocks where the sciatic nerve crosses the piriformis muscle, causing pain that starts at the point of contact and that extends down the back of the leg. Pain can also occur with pelvic or rectal examination.

Patients are frequently advised to stop running, bicycling, or performing any activity that elicits pain. A patient whose pain is aggravated by sitting should stand up immediately or, if unable to do so, change positions to raise the painful area from the seat. Stretching exercises, although often recommended, are rarely beneficial, and any movement that raises the knee forcibly often aggravates symptoms. A corticosteroid injection into the site near where the piriformis muscle crosses the sciatic nerve often helps, presumably by reducing fat around the muscle, making it less likely to press on the nerve.

Thoracic Outlet Compression Syndromes

A Group of Syndromes Characterized by Symptoms of Pain and Paresthesias in the Hand, Neck, Shoulder, or Arms.

These syndromes include the neurovascular compression syndromes of the shoulder girdle, scalenus anticus syndrome, and cervical rib syndrome. They are experienced more commonly in women, usually between ages 35 and 55. More specifically, thoracic outlet syndrome (TOS) is due to compression/irritation of brachial plexus (BP) elements (“neurogenic TOS”) and/or subclavian vessels (“vascular TOS”) in their passage from the cervical area toward the axilla. The usual site of entrapment is the interscalenic triangle. TOS is a source of disagreement among clinicians regarding its incidence, diagnostic criteria and optimal treatment. Constitutional factors, like a cervical rib, predispose to the development of TOS. The syndrome often develops during the 3rd or 4th decade, following external factors such as trauma, weight excess, incorrect shoulder posture.

The clinical picture can be varied: pain in the cervical region and arm, paresthesias (medial side of arm predilected) aggravated by overhead positions of the arms, hand intrinsic muscle deficit/atrophy, easy fatigability, paleness, coldness of hand. The clinical examination may be entirely normal or show cervical muscle spasm, tenderness of BP in the supraclavicular area, radial pulse attenuation and occurence of symptoms upon positional maneuvers, sensory or motor deficit. The diagnosis is based upon clinical evaluation and absence of other relevant pathology. Therefore, the cervical spine and distal peripheral nerves are studied by radiological and electrophysiological studies. There is no laboratory test confirming TOS: most of the time, there is no anatomic variation seen radiologically and electrophysiological testing is normal. The scalene muscle block appears a helpful diagnostic tool if used with the other clinical data.

The distribution of symptoms suggests the syndrome. Symptoms of pain and paresthesias are most often distributed medially in the arms and sometimes extend into the adjacent anterior chest wall. Many patients have mild to moderate sensory impairment in the C-8 to T-1 distribution on the painful side, and a few have prominent vascular-autonomic changes in the hand, including cyanosis, swelling, and (rarely) Raynaud's phenomenon or distal gangrene.

Unless there is significant motor deficit or subclavian artery compression, the treatment should be kept conservative as long as possible. However in case of neurological deficit or symptoms unresponsive to medical treatment, the patient may be advantageously treated according to the present invention with botulinum toxin type B introduced into the area of nerve compression and/or inflammation.

The recommended initial dose of MYOBLOC™ for patients with a prior history of tolerating botulinum toxin injections is 2500 to 5000 U divided among affected muscles for the treatment of spasmodic torticollis. Dosages for other indications are adjusted up or down, depending on the volume of muscle or perineural area to be denervated. A second administration may also be made if the clinical effects of the first injection(s) are not as strong as expected. Patients without a prior history of tolerance to botulinum toxin injections should receive a lower initial dose. Subsequent dosing is titrated according to the patient's individual response.

The method described for performing the potency assay is specific to Elan Pharmaceutical's manufacture of MYOBLOC™. Due to differences in the specific details of this assay such as the vehicle, dilution scheme and laboratory protocols for various potency assays, Units of biological activity of MYOBLOC™ cannot be compared to or converted into units of any other botulinum toxin or any toxin assessed with any other specific assay method. Therefore, differences in species sensitivities to different botulinum neurotoxin serotypes preclude extrapolation of animal dose-activity relationship to human dose estimates. However, the selection and proper administration dosage is within the skill of the ordinary physician who has skill in treatment with neurotoxins. The duration of effect in patients responding to MYOBLOC™ treatment has been observed in studies to be between 12 and 16 weeks at doses of 5000 U or 10,000 U. (More detailed dosage and administration information is available, for example in Physician's Desk Reference, 2002 Edition under MYOBLOC™ (Elan) (Botulinum Toxin Type B).

There are a variety of methods for positioning a needle into the deep muscle tissue or into the tunnel structures that are the root cause of the entrapment syndromes. Some of the methods available are electromyography, computed tomography and magnetic resonance imaging, see for example, Britz G W, Dailey A T, et al., Magnetic resonance imaging in the evaluation and treatment of peripheral nerve problems. Perspectives in Neurosurgery 6:53-66 (1995), which is hereby incorporated by reference in its entirety. Additionally, arthroscopic techniques allow exploration and introduction of therapeutics into the area around joints such as the elbow.

By way of non-limiting example, the piriformis muscle is a relatively small structure located as far as eight inches below the surface of the buttock. If a blind injection misses the muscle, or strikes the sciatic nerve or the colon it may lead to significant complications. The use of Open MRI image guidance allows the administering physician to perform a reliable and accurate procedure. Flash MRI images each take about 12 seconds to complete and allow viewing of the progress and angle of approach of the needle into the deep tissue. Before this invention, physicians would inject a long-lasting local anesthetic and a steroid to relieve inflammation. However, using the present invention, the physician employs this technique to place the needle into and/or around the piriformis muscle to administer a therapeutically effective amount of botulinum toxin B. The patient then benefits from both the muscle relaxing effects as well as the pain relief provided thereby.

While the invention has been described with reference to specific methods and embodiments, it will be appreciated that various modifications and changes may be made without departing from the invention.

It is claimed:

1. A method of treating a nerve entrapment syndrome in a patient in need of such treatment by administering a therapeutically effective amount of Botulinum toxin type B to the patient.

2. A method of reducing pain caused by a nerve entrapment syndrome in a patient in need of such treatment by administering a therapeutically effective amount of Botulinum toxin type B to the patient.

3. The method of claim 1 or claim 2 wherein the toxin is administered via injection through a needle.

4. The method of claims 1 or 2 wherein the compression syndromes are selected from the group consisting of piriformis syndrome, carpal tunnel syndrome, cubital tunnel syndrome, and radial tunnel syndrome.

5. The method of claim3 wherein the injection is made into muscle tissue impinging on a compressed nerve.

6. The method of claim 3 wherein the injection is made into the interstitial area around a compressed nerve.

7. The method of claim 3 wherein the injection is made into connective tissue surrounding the compressed nerve.
